(19) United States
(12) Patent Application Publication
(10) Pub. No.: US 2018/0008804 A1
(43) Pub. Date: Jan. 11, 2018

(54) A FOLDING DEVICE TO ASSIST IN SELF INSERTION OF A CATHETER TUBE INTO THE URETHRAL ORIFICE OF WOMEN

(71) Applicant: GR Dome Medical Ltd., Tirat Carmel (IL)

(72) Inventor: Amir LANIADO, Haifa (IL)

(73) Assignee: GR Dome Medical Ltd., Tirat Carmel (IL)

(21) Appl. No.: 15/544,257

(22) PCT Filed: Jan. 20, 2015
(86) PCT No.: PCT/IL2015/000002

§ 371 Date: Jul. 18, 2017

(51) Int. Cl. A61M 25/01; A61M 25/00

(52) U.S. Cl. CPC A61M 25/01; A61M 25/0017; A61M 2210/1092

(57) ABSTRACT

The present invention is a folding urethral catheter tube insertion device for women, abbreviated to: FUCID. The device facilitates easy finding of the location of the external urethral orifice in women and assists in the self insertion of a catheter tube into the urethra orifice while widening the orifice. Once inserted, user is able to disconnect the device from her body while maintaining the catheter tube in the urethra tract. The FUCID is typically produced as a single entity product and is typically produced by injection cast molding technology of a semi-rigid material. The FUCID folds to a compact, small size configuration, that is suitable and convenient to carry around in a small purse or small bag. The folded configuration of the FUCID is easily and simply unfolded to a ready-for-deployment configuration. The device is is constructed of: a vaginal insertion portion, abbreviated to VIP, a hollow funnel shaped tube guiding channel portion, abbreviated to TGCP, and a bridging plate between the two portions. The bridging plate is connected in a fixed connection of approximately 90 degrees to the widen side of TGCP and is connected to the VIP by a flexible hinge connection. Flexible wing curved bars protrude from the TGCP above the plain of the connected bridging plate. In its folded configuration of the FUCID, the VIP is folded to be in an approximate spatial parallel configuration with TGCP. In the FUCID unfolded configuration, the VIP is folded to be in an approximate aligned spatial configuration with FUCID. In deploying the FUCID, the TGCP aligned VIP is inserted into the vagina while positioning the opening of the wide side of TGCP over the uretheral orifice of the patient. The TGCP is pushed towards the body of the patient, causing the flexible wing curved bars to come into contact with the skin surrounding the uretheral orifice and to widen the orifice opening. With orifice openened the catheter tube is inserted through the TGCP.

### FIELD OF THE INVENTION

The present invention is a folding device to assist in the insertion of a catheter tube into the urethral tract of women. More specifically, the folding device of the present invention is compact and easy to pack for carrying around. The device simplifies the finding of the location of the external urethral orifice and widens the orifice so that women patients may perform self-insertions of a catheter tube into the external orifice of the urethral tract without the assistance of a physician or a caregiver.

### BACKGROUND OF THE INVENTION

The term catheter is defined by the Merriam-Webster dictionary (http://www.merriam-webster.com/dictionary/catheter) as: a thin tube that is put into the body to remove or inject a liquid or to keep a passage open. In the context of the present invention the terms: “tube”, “catheter” and “catheter tube”, refer interchangeably to a thin tube that is inserted through the external orifice of the urethra of women and is utilized for the input of medication into the urethra tract and/or bladder and/or for the removing of urine from the bladder. In the text that follows the device of the present invention to assist in the insertion of a tube into the urethral tract in women is also reffered to as: “the urethra catheter applicator”, or, interchanchably, as: “the urethra guide”.

Conditions such as cystitis, bladder pain syndrome, interstitial cysttitis, urethritis, urethra pain syndrome are inflammatory conditions of the submucosal and muscular layers of the bladder without infectious pathogens. The cause of lower urinary tract inflammation is often, but not necessarily, triggered by an infection (bacterial cystitis) is unknown in many patients and the condition is regarded as a diagnosis of exclusion. Inflammation of the lower urinary tract inflammatory conditions is associated with urinary urgency, urinary frequency, waking at night to urinate (nocturia), and pain/discomfort. Symptoms may overlap with other urinary bladder disorders such as: urinary tract infection (UTI), overactive bladder and prostatitis.

Bladder instillation of pharmaceutical agents is one of the main forms of treatment of lower urinary tract inflammation. Advantages of this treatment approach include direct contact of the medication with the bladder wall and decreased risk of systemic side effects. Bladder instillations use a transurethral approach where a drug is instilled directly into the bladder using a catheter tube on several occasions. Because of the unaccessible position of the urethra in women it is difficult for most women patients to self-administer the bladder instillation.

Patients that suffer from urinary retention problems or from inability to completely empty their bladder during urinating are often treated intermittently with a catheter. The ability to self-insert a catheter tube through the urethra orifice frees a treated patient of relaying on assistance from a medically trained person which is not always convenient or available. Self-insertion of a catheter tube requires fine motor skills and a good vision to locate and penetrate the urethra orifice.

In women, the urethra tract is short compared to males and located at the bottom of the pelvis. The urethra tract is about 3.5-5 cm long and exits the body between the clitoris and the vagina. The women external urethral orifice is located 1-2 cm below the clitoris behind the symphysis pubis (also referred to as: pubic bone. The entire length of the urethra tract is embedded in the anterior vaginal wall and it is slightly curved with the concavity directed downwards. Because of the anatomic boundaries many patients requiring bladder instillations find it difficult to pass the catheter through the urethra tract into the bladder and most local treatment regimes require the patient to return to the physician or trained caregiver repeatedly or undergo training to manage it by themselves.

Prior art devices for self-insertion of catheter-tubes are commonly constructed of two connected portions: a vaginal insertion portion and a catheter tube guide portion. The vagina inserted portion stabilizes and maintains the catheter tube guiding portion in a placement which enables the insertion of the catheter tube through the hole into the urethra orifice. Examples of self-insertion devices of a catheter tube for women that have two portion units are given in U.S. Pat. No. 5,045,078 by Asta, U.S. Pat. No. 5,084,036 by Rosenbaum and PCT/IL2014/000051 by Laniado.

PCT/IL2014/000051 (Laniado) discloses a device to assist in the insertion of a catheter tube into the urethral tract of women that has a configuration that is structured of an elongated plate-shaped vaginal insertion portion and an elongated plate-shaped catheter tube guiding portion. The vaginal insertion portion has a configuration to be placed adjacent to the pubic symphysis of women. At least two wing structures connect and protrude from the plate-shaped catheter tube guiding portion and are configured to widen the urethral orifice of a treated patient. The catheter tube guiding portion has a hole at its unconnected edge through which a catheter tube is inserted into said orifice of urethral tract. The vaginal insertion portion and the catheter tube guiding portion connect in a spatial configuration that forms a rigid, approximate L shaped structure. The L shaped structure connects to a vaginal insertion element that serves to push the plate-shaped vaginal insertion portion into the vagina when the device is deployed. Two embodiments are described for the vaginal insertion portion: a rod-handle and a tumble. The vaginal insertion element is fixated to the vaginal insertion portion, or alternatively, is reversibly connected to the vaginal insertion portion.

The approximate L shaped rigid structure, together with the fixated vaginal insertion element described in PCT/IL2014/000051 form a device that maybe unsuited to be carried in a small purse or small-size hand bag. When the vaginal insertion element is not fixated to the L shaped structure, it becomes necessary to carry two separate components: the L shaped structure and the vaginal insertion element. The necessity to put the two components together to deploy the device add to the bulkiness of carrying the L shaped structure device.

### SUMMARY OF THE INVENTION

In the context of the present invention the terms “tube”, “catheter” and “catheter tube” are used interchangeably and refer to a thin tube that is inserted through the external orifice of the urethra of women and is utilized for the input of medication into the urethra tract and/or bladder and/or for the removing of urine from the bladder.

The present invention is a device for assisting in the insertion of a catheter tube into the external orifice of the urethral tract in women. The device is referred interchangeably in the text that follows as the: folding urethral catheter insertion device, abbreviated to: FUCID.

The FUCID is typically produced as a single entity product typically produced by injection cast molding technology of a semi-rigid material such as, but not limited to silicon, plastic or nylon material.

The FUCID folds to a compact, small size configuration, that is suitable and convenient to carry around in a small purse or small bag. The folded configuration of the FUCID is easily and simply unfolded to a ready-for-deployment configuration.

The compact configuration of the FUCID enables the efficient pre-packing of a FUCID as an integral part of small-volume disposable catheter tube kit for a single, one-time use. The small-volume of FUCID catheter tube kits are convenient for storing in pharmacies, hospitals, clinics and for home use and provide women that require repeated insertion of a catheter-tube through their uretheral orifice with the option of carrying a number of disposable catheter tube kits in discretion in small size purses or small size bags.

The FUCID facilitates easy finding of the location of the external urethral orifice and assists in the procedure of the insertion of a catheter into the urethra orifice while widening the orifice. In addition, the FUCID of the present invention enables the user to disconnect the device from her body while maintaining the catheter tube in the urethra tract after it has been inserted.

The FUCID of the present invention enables women to perform by themselves the instillation of medicinal substances for the treatment of inflammation along the urethral tract bladder and/or removal of urine from the bladder without the assistance of a physician or a trained caregiver while substantially reducing the discomfort caused to patients in the course of preforming the medical procedure.

The FUCID of the present is constructed of structure that has:

a vaginal insertion portion, abbreviated as: VIP, a hollow funnel shaped tube guiding channel portion, abbreviated as: TGCP and a bridging plate.

The VIP is an elongated plate structure designed to be pressed in the width dimension towards the edge of the vagina, in the clitoris direction.

The TGCP is a round hollow cylinder that gradually diminishes in its circumference from one end to the other, and has an opened channel slit, opened on both its ends, running through its entire length. The opened channel slit is designed to enable the movement of a catheter tube into and out of the opened channel slit as well as to remove a catheter tube from the opened channel slit moving (by lifting) the catheter tube through the lips of the channel slit.

On the wide side of the TGCP, surrounding the opening of the channel slit, are at least two protruding thin flexible wing curved bars configured to contact the skin surrounding the urethral orifice of women and to widen the urethral orifice of women when said hollow funnel shaped tube guiding channel is pressed towards the orifice. The Merriam-Webster dictionary (http://www.merriam-webster.com) after defining “wing” as a part of an animal's body that is used for flying or gliding also defines the term “wing” as an appendage or part resembling a wing in appearance, position, or function.

The bridging plate of the FUCID has a flat plate configuration, and is connected in a fixed connection of approximately 90 degrees to the widen side of the TGCP at its opposite edge, in a, folding, hinge connection, to an edge of the VIP.

The TGCP of the FUCID has on its wide side at least two protruding thin flexible wing curved bars configured to contact the skin surrounding the urethral orifice of women and to widen the opening of the urethral orifice of women when the hollow funnel shaped tube guiding channel is pressed towards the orifice. Optionally, the thin flexible wing curved bars have teeth at their edges to stabilize the reversible contact between the flexible wing curved bars and the skin surrounding the urethral orifice.

In a folded configuration of the FUCID, the VIP is folded to be in an approximate spatial parallel configuration with the TGCP,

In an unfolded configuration of the FUCID, the VIP is folded to be in an approximate aligned spatial configuration with the TGCP.

Optionally, the plate of the VIP of the FUCID has a cut slit that runs the plate from the an edge of the plate to a short distance from its edge. The bridging plate of the FUCID has a curved bar protruding from the center of its flat plate.

In an unfolded configuration of the FUCID, the VIP is folded to be in an approximate aligned spatial configuration with the TGCP. The curved bar protruding of the bridging plate is inserted through the cut slit of the plate of the VIP and reversibly connects to the edge of said cut slit of the VIP. With the curved bar reversibly connected to the edge of the slit in the VIP, the VIP is fixated.

Optionally, the plate of VIP has a protruding latching bar and the hollow funnel shaped tube guiding channel portion has a protruding connection bar with a pointed-bar section. In a folded configuration the FUCID the VIP is folded to be in an approximate parallel spatial configuration with the TGCP. The protruding connection bar with a pointed-bar section reversibly connects with the protruding latching bar, thus the folded configuration of the FUCID is reversibly fixated.

### BRIEF DESCRIPTION OF THE DRAWINGS

In order to better understand the present invention, and appreciate its practical applications, the following Figures are provided and referenced hereafter. It should be noted that the Figures are given as examples only and in no way limit the scope of the invention. Like components are denoted by like reference numerals.

FIG. 1 is an isometric illustration of from-the-top view of a FUCID of the present invention in a folded, configuration.

FIG. 2 is an illustration of a crosscut view from the side of the FUCID shown in FIG. 1, shown in a folded, configuration.

FIG. 3 is an isometric illustration of from-the-top view of a FUCID of the present invention in an un-folded configuration.

FIG. 4 is an illustration of a crosscut view from the side of the FUCID shown in FIG. 3, shown in an un-folded configuration.

FIG. 5 to and including FIG. 11 illustrate consecutive stages in the deploying of the FUCID illustrated in FIG. 1 up to and including FIG. 4.

FIG. 5 illustrates the patient holding the FUCID of the present invention, in both hands, with the FUCID in a folded configuration as shown in FIG. 1 and FIG. 2.

FIG. 6 illustrates the patient un-folding the FUCID illustrated in FIG. 5 (the unfolded configuration shown in FIG. 3 and FIG. 4).

FIG. 7 illustrates the inserting a catheter tube into the FUCID illustrated in FIG. 6.

FIG. 8 illustrates the catheter tube inserted into the unfolded FUCID, shown in FIG. 7 and the FUCID positioned over the urethral orifice of the treated patient (the urethral orifice not shown).

FIG. 9 illustrates the FUCID, shown in FIG. 8, pushed towards the body of the patient to form contact with the skin surrounding the urethral orifice of the treated patient (the urethral orifice not shown) and the patient pushing the catheter tube, shown in FIG. 8, pushed by the patient into her urethral tract.

FIG. 10 illustrates the removing of the catheter tube from the catheter tube lead-channel of the FUCID, both illustrated in FIG. 8, thus, separating the catheter tube from the FUCID.

FIG. 11 illustrates the catheter tube, illustrated in FIG. 8 and FIG. 9 inserted into the urethral tract of the treated patient and the FUCID, shown in FIG. 8 and FIG. 9, removed from the body of the treated patient.

FIG. 12 is a cross cut, from the side, schematic illustration of internal organs of a treated patient with the FUCID shown in FIG. 3 deployed in her body.

FIG. 13A illustrates a schematic cross cut, from the front view, of the FUCID shown in FIG. 3 and FIG. 4, deployed in a treated patient, showing initiation of contact with the skin surrounding the urethra orifice of the patient.

FIG. 13B is a schematic cross cut illustration of the FUCID shown in FIG. 13A, showing the contact with the skin surrounding the urethra orifice of the FUCID and widening of the orifice of the urethra of the treated patient.

### DETAILED DESCRIPTION OF AN EMBODIMENT OF THE PRESENT INVENTION

To describe an embodiment of the present invention of the FUCID (abbreviation of: “folding urethral catheter insertion device”): reference is presently made to the figures.

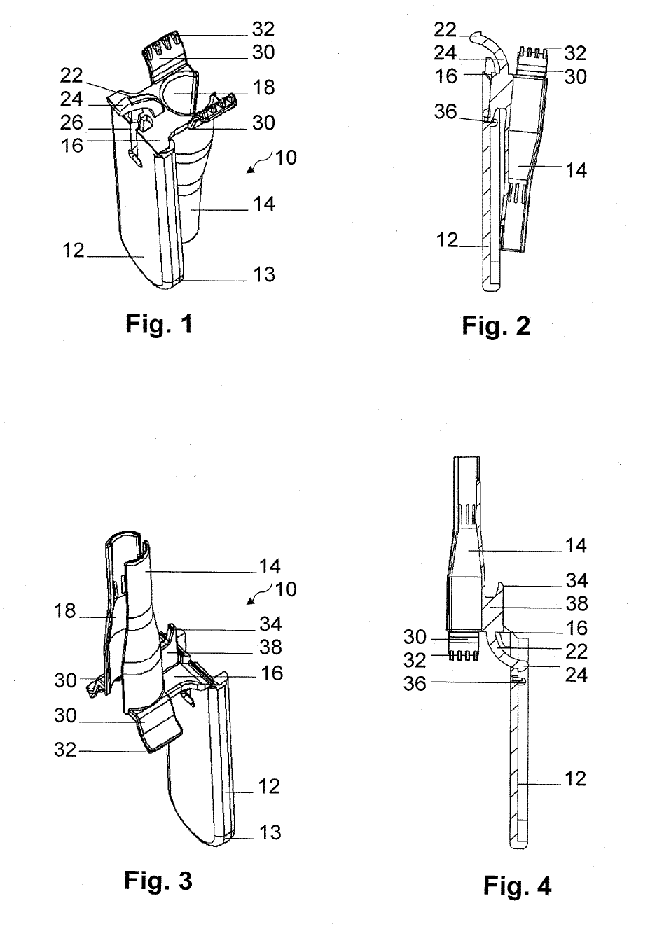

In FIG. 1 to and including FIG. 4, reference is made to the construction of the FUCID (10) of the present invention.

FIG. 1 and FIG. 3 are isometric illustrations from-the-top view of a FUCID (10) of the present invention in a folded and unfolded configurations, respectively.

FIG. 2 FIG. 4 are crosscut, from-the side view, illustrations of the FUCID (10) of the present invention in a folded and unfolded configurations, respectively.

The FUCID (10) is divided into two portions connected together by a flat bridging plate (16).

The first portion is a plate portion, typically, but not limited to, an elongated rectangle configuration, referred to as the vaginal insertion portion (12) abbreviated as: VIP. The second portion is an elongated, cylinder hollow funnel structure tube that gradually becomes smaller in its circumference with a channel-slit portion running through it, referred to as the: catheter tube guiding channel portion (14), abbreviated as: TGCP. One of edge the VIP (12) and the wide edge of TGCP (14) are connected via the flat bridging plate (16). The connection of TGCP (14) to flat bridging plate (16) is a rigid, approximately 90 degrees connection. The connection of the VIP (12) to bridging plate (16) is a flexible hinge-connection that enables the free moving of VIP (12) so as to position the VIP (12) in parallel to the TGCP (14) when the FUCID (10) is in the folded configuration (shown in FIG. 2), and to position the VIP (12) in an approximate alignment with the TGCP (14) (an approximately 180 degrees angle folding from the parallel configuration) when the FUCID (10) is in an unfolded configuration (shown in FIG. 4).

In the unfolded configuration of FUCID (10), the distance between the plate surface of VIP (12) and the edge of the rim of the opening of the guiding channel (18) in TGCP (14), confined by flat bridging plate (16) is typically, but not limited to, 2 to 14 millimeters.

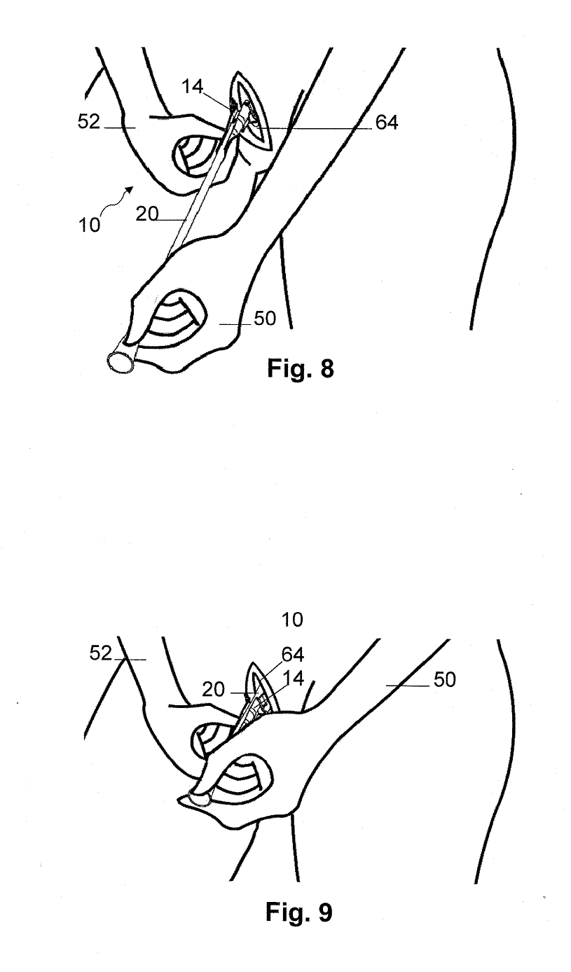

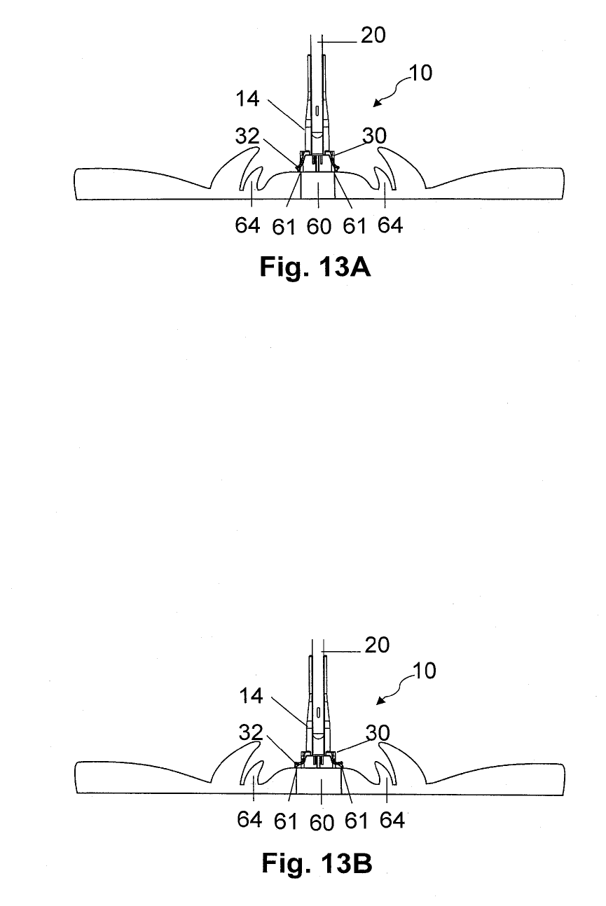

The free edge of the plate of the VIP (12) is typically rounded (13) so as to minimize the inconvenience when the VIP is inserted into the vagina of a treated patient. The opened channel-slit running along the full length of the TGCP (14) is referred to as the catheter tube guiding channel (18). Guiding channel (18) is designed to enable the insertion of a catheter tube (20) into and out of the channel, as shown FIG. 7 and FIG. 9. The resilience of the construction-material of TGCP (14) and the distance between the two parallel lips of channel (18) enables the removal of catheter tube (20) from the TGCP (14) by moving the catheter tube (20) sideways (lifting it perpendicularly) from channel (18), as illustrated in FIG. 8. Bridging plate (16) has a flexible curved bar (22) protruding from the plate towards VIP (12) (shown in FIG. 2). Bar (22) has a bulging-hook (24) at its end, VIP (14) has a slit (26) in its plate that enables bar (22) to penetrate through the plate. At the widen edge of TGCP (14), protruding from both sides of the channel (18) of the TGCP (14) and over flat bridging plate (16) are at least two thin flexible wing curved bars (30). Flexible wing curved bars (30) curve in the direction away from channel (18) and optionally, have teeth (32) at their edges. Wing curved bars (30) are configured so as widen the orifice of the urethra tract when pressed to the skin surrounding orifice, as shown in FIGS. 13A and 13B. Teeth (32) assist in stabilizing the reversible connection between wing curved bars (30) and the skin surrounding the urethral orifice.

Optionally, on the other side of curved bar (22), protruding from bridging plate (16), is a connection bar (38) with a pointed-bar section (34). Below slit (26) in VIP (12), in the direction of the free edge of the plate of the VIP (13) and on the side facing TGCP (14) in the closed configuration of FUCID (10), is a protruding semi-circle latching bar (36) that reversibly surrounds and latches to pointed-bar section (34), shown in FIG. 2. When the FUCID (10) is in a folded configuration, the reversible connection between connection pointed bar section (34) of connection bar (38) and semi-circle latching bar (36) maintain VIP (12) and TGCP (14) stably and reversibly connected.

In unfolding FUCID (10), pointed-bar section (34) of connection bar (38) is disconnected from protruding semi-circle latching bar (36) and the position of the VIP (12), using the hinge connection with bridging plate (16), is changed so as to be in alignment with TGCP (14). With the movement of VIP (12), curved bar (22) is inserted into slit (26) in VIP (12) and a bulging-hook (24) reversibly connects to the edge of slit (26), thus, reversibly fixates the unfolded connection configuration between VIP (12) and TGCP (14), shown in FIG. 3 and FIG. 4. In the fixated configuration VIP (12) is in an alignment position to the length of channel (18) of TGCP (14).

Reference is presently made to consecutive stages of deploying the FUCID (10) of the present invention.

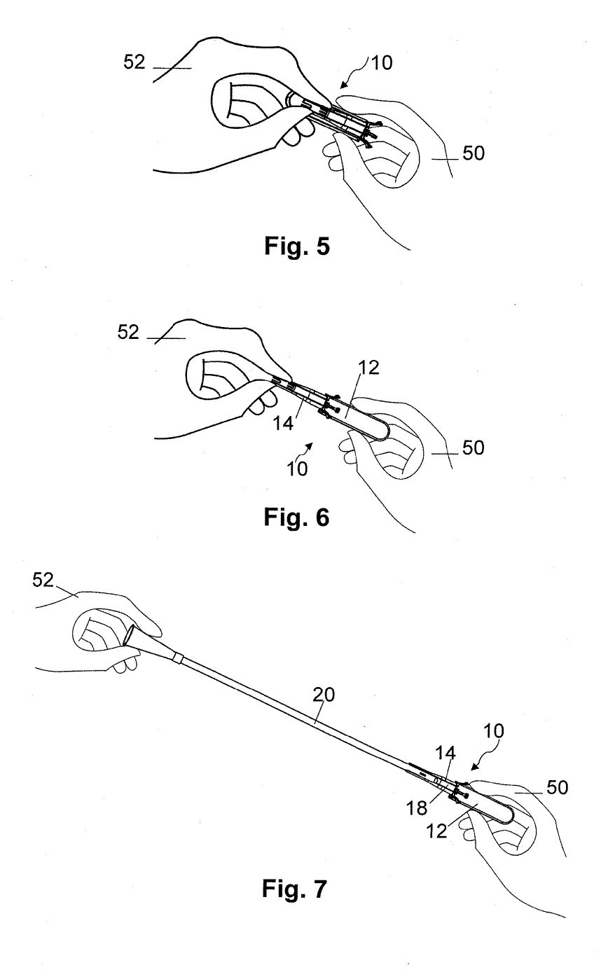

FIG. 5 illustrates the patient holding the FUCID (10) in a folded configuration with both hands ((50) and (52)). FIG. 6 illustrates the patient, using both hands ((50) and (52)) un-folding the FUCID (10) by moving (folding) VIP (12) from TGCP (14). The unfolded configuration is reversibly fixated by the connection of bulging-hook (24) to the edge of slit (26), as was previously explained. FIG. 7 illustrates the inserting by one hand of the patient (52), a catheter tube (20) into channel (18) of TGCP (14) while the other hand (50) holds VIP (12) of FUCID (10) in an unfolded configuration. The FUCID (10) is now ready to be inserted into the body of the treated patient.

FIG. 8 illustrates the patient with one hand (52) positioning the FUCID (10) over her urethral orifice and the other hand (50) holding catheter tube (20). In FIG. 8 the patient is shown holding TGCP (14) with VIP (12) inside her vagina (the urethral orifice and vagina not shown in the Fig). VIP (12) is inserted into the vagina by pushing the connected and aligned TGCP (14) towards the body of the patient. With the VIP (12) tightly pressed against the wall of the vagina and adjacent to the pubic symphysis of the woman, and the wing-bars (30) of TGCP (14) in contact with the skin surrounding the urethral orifice and widen the orifice, catheter tube (20) is inserted into the urethral tract of the patient. FIG. 9 illustrates the patient holding the TGCP (14) with one hand (52) and with the other hand (50) pushing tube (20) through TGCP (14) and through the orifice of the urethral tract into her urethral tract.

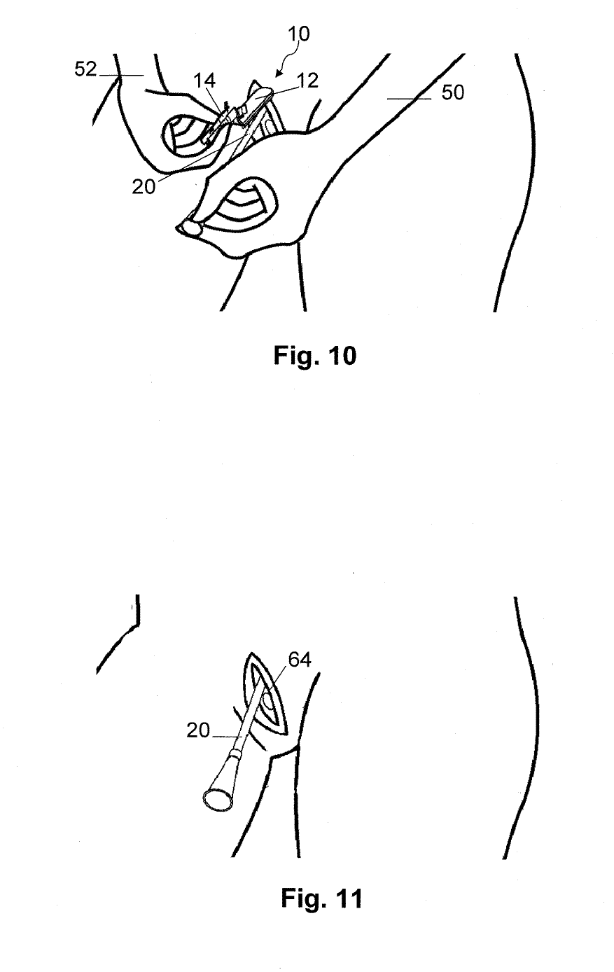

With catheter tube (20) inside the urethral tract of the patient, FIG. 10 illustrates the removing of catheter tube (10) from channel (18) of the TGCP (14). In the Fig. the patient hold tube (20) with one hand (50) while the other hand (52) holds the TGCP (14) of the FUCID (10) with VIP (12) shown outside the vagina and FUCID (10) is disconnected from catheter tube (20). FIG. 11 illustrates catheter tube (20), inserted into the urethral tract of the treated patient and FUCID (10) no longer part of “the scene”.

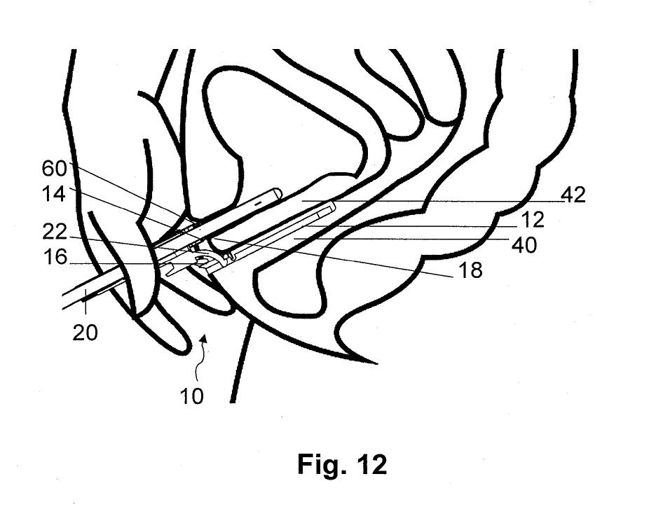

Reference is presently made to FIG. 12.

FIG. 12 is a cross cut, from the side, schematic illustration of internal organs of a treated patient with FUCID (10) deployed in her body.

FIG. 12 illustrates VIP (12) of FUCID (10) inserted into the vagina (40) and pressed against the pubic symphysis (42) of the treated patient. Bridging plate (16) is illustrated connecting TGCP (14) with VIP (12) in a reversibly fixated configuration by flexible curved bar (22). Catheter tube (20) is illustrated inserted through channel (18) of TGCP (14), through the urethral orifice (60) and into the bladder of the patient.

Reference is presently made to FIG. 13A and FIG. 13B.

FIG. 13A illustrates a schematic cross cut, from the front, view, of the FUCID (10) deployed in a treated patient, showing initiation of contact with the skin surrounding the urethra orifice of the patient. FIG. 13B is a cross cut illustration of the FUCID (10) shown in FIG. 13A, showing the contact with the skin surrounding the urethra orifice (61) and widening of the orifice of the urethra of the treated patient.

In deploying FUCID (10), TGCP (14), in the unfolded configuration of the FUCID (10), is pressed towards the body of the patient, causing the connected VIP (12) to be inserted into the vagina (40) through the upper section of the labia minora lips (64). The VIP (12) is pushed towards the pubic symphysis (42) so that the opening of channel (18) of the TGCP (14) is positioned over the orifice of the urethral tract (60). The distance of the bridging between the VIP (12) and the TGCP (14) by bridging plate (16), typically 2 to 14 millimeters, enables the positioning of the VIP(12) stabilized TGCP (16) at just the desired location. Catheter tube (20) at this stage is inserted into channel (18) but does not protrude from the channel (shown in FIGS. 9 and 13A). In pressing TGCP (14) towards the body of the patient teeth (32) of flexible wing-bars (30) contact the skin surrounding the urethral orifice (60), as illustrated in FIG. 13A (and FIG. 8)

Continuing to press TGCP (14) towards the body of the patient causes the curved flexible wing-bars (30), connected to the skin surrounding the urethral orifice (61), optionally by teeth (32), to pull away the skin away from the orifice. The motion of the skin surrounding the urethral orifice widens the diameter of the opening of the orifice, as illustrated in FIG. 12B With the orifice's opening widened, catheter tube (20) is pushed through channel (18) and into the urethral tract through the widened urethral orifice, as illustrated in FIG. 9.

What is claimed:

1) A folding device for assisting in the insertion of a catheter tube into the external orifice of the urethral tract of women, comprising:
a structure, comprising:
a vaginal insertion portion
a hollow funnel shaped tube guiding channel portion,
a bridging plate,
wherein, said vaginal insertion portion comprises an elongated plate structure designed to be pressed in the width dimension towards the edge of the vagina, in the clitoris direction,
wherein, said hollow funnel shaped tube guiding channel portion comprises a hollow round cylinder that gradually diminishes in its circumference from one end to the other and has an opened channel slit, opened on both its ends, running through its entire length,
wherein, said opened channel slit of a hollow funnel shaped tube guiding channel portion is designed to enable the movement of a catheter tube into and out of the said opened channel slit,
wherein, said opened channel slit of a hollow funnel shaped tube guiding channel portion is designed to enable the removal of a catheter tube from said opened channel slit by moving the catheter tube through the lips of said opened channel slit,
wherein said hollow funnel shaped tube guiding channel portion has on the wide side of said funnel shaped tube guiding channel portion, surrounding the opening of the channel slit, at least two protruding thin flexible wing curved bars configured to contact the skin surrounding the urethral orifice of women and to widen the urethral orifice of women when said hollow funnel shaped tube guiding channel is pressed towards the orifice,
wherein said bridging plate has a flat plate configuration,
wherein, an edge of said bridging plate is connected in a fixed connection of approximately 90 degrees to the widen side of said hollow funnel shaped tube guiding channel portion and the at its opposite edge, said bridging plate is connected in a, folding, flexible hinge connection, to an edge of said vaginal insertion portion,
wherein, said thin flexible wing curved bars protrude from said hollow funnel shaped tube guiding channel portion, above the plain of connected said bridging plate,
wherein, in a folded configuration, said folding device for assisting in the insertion of a catheter tube into the external orifice of the urethral tract of women, said vaginal insertion portion is folded to be in an approximate spatial parallel configuration with said hollow funnel shaped tube guiding channel portion,
wherein, in an unfolded configuration, said folding device for assisting in the insertion of a catheter tube into the external orifice of the urethral tract of women, said vaginal insertion portion is folded to be in an approximate aligned spatial configuration with said hollow funnel shaped tube guiding channel portion.

2) The thin flexible wing curved bars of the hollow funnel shaped tube guiding channel portion of the device as set forth in claim 1, wherein, said thin flexible wing curved bars have teeth at their edges.

3) The bridging plate of the device as set forth in claim 1, wherein the length of said bridging plate between the connection of the vaginal insertion portion and the hollow funnel shaped tube guiding channel portion is between 2 and 14 millimeters.

4) The vaginal insertion portion of the device as set forth in claim 1, wherein, said vaginal insertion portion has a cut slit that runs through said plate from the an edge of the plate to a short distance from the edge.

5) The flat bridging plate of the device as set forth in claim 1, wherein, said bridging plate has a curved bar protruding from the center of the plate.

6) In an unfolded configuration of the device as set forth in claim 1, wherein said vaginal insertion portion is folded to be in an approximate aligned spatial configuration with said hollow funnel shaped tube guiding channel portion, wherein curved bar protruding of the said flat bridging plate set forth in claim 3 is inserted through the cut slit of said vaginal insertion portion, set forth in claim 2, and reversibly connects to the edge of said cut slit of said vaginal insertion portion.

7) The vaginal insertion portion of the device as set forth in claim 1, wherein, said vaginal insertion portion has a protruding latching bar.

8) The hollow funnel shaped tube guiding channel portion of the device as set forth in claim 1, wherein, said hollow funnel shaped tube guiding channel portion has a protruding connection bar with a pointed-bar section.

9) In a folded configuration of the device as set forth in claim 1, wherein said vaginal insertion portion is folded to be in an approximate parallel spatial configuration with said hollow funnel shaped tube guiding channel portion, wherein protruding connection bar with a pointed-bar section, set forth in claim 6, reversibly connects with protruding latching bar, set forth in claim 5.

10) The structure of the device as set forth in claim 1, wherein the structure is a single entity structure.

11) The structure of the device as set forth in claim 1, wherein the structure is produced by injection cast molding.

12) The vaginal insertion portion, the hollow funnel shaped tube guiding channel portion and the bridging plate, of the device as set forth in claim 1, wherein said vaginal insertion portion, said hollow funnel shaped tube guiding channel portion and said bridging plate, are made of semi rigid material.
